(19) United States
(12) Patent Application Publication
(10) Pub. No.: US 2026/0034145 A1
(43) Pub. Date: Feb. 5, 2026

(54) APPLICATION OF CONESSINE IN PREPARING ANTI-INFLUENZA A VIRUS INFECTION DRUG

(71) Applicant: Shanghai Jiaotong University School of Medicine, Shanghai (CN)

(72) Inventors: Qiming Liang, Shanghai (CN); Jingsong Jiao, Shanghai (CN); Xin Wang, 200025 (CN)

(21) Appl. No.: 19/285,594

(22) Filed: Jul. 30, 2025

(30) Foreign Application Priority Data
Aug. 2, 2024 (CN) 202411055977.3

(51) Int. Cl. A61K 31/58; A61P 31/16

(52) U.S. Cl. CPC A61K 31/58; A61P 31/16

(57) ABSTRACT

The present disclosure provides an application of Conessine in preparing an anti-influenza A virus (IAV) infection drug, falling within the technical field of biomedicine. In the present disclosure, the small molecule Conessine has a strong inhibitory effect against IAV, IC₅₀=0.45 μM, with a low numerical value and high inhibitory efficiency. In the present disclosure, the toxicity of Conessine to cells is detected, it is found that the toxicity of Conessine to cells is also low, CC₅₀ is high, a selection index (SI) is much higher than a safe range, and the safety and effectiveness of Conessine as an IAV inhibitor are more guaranteed. Conessine can be used for treating respiratory infections, pneumonia, encephalitis and other diseases caused by IAV, providing a new effective treatment drug for global influenza epidemic.

### CROSS-REFERENCE TO RELATED APPLICATION

This application claims priority of Chinese Patent Application No. 202411055977.3, filed on Aug. 2, 2024, the entire contents of which are incorporated herein by reference.

### TECHNICAL FIELD

The present disclosure relates to the technical field of biomedicine, and in particular to an application of Conessine in preparing an anti-influenza A virus (IAV) infection drug.

### BACKGROUND

IAV is a human respiratory pathogen, which is widely prevalent all over the world, with the characteristics of strong infectivity and high pathogenicity. It can cause severe respiratory infections, pneumonia, encephalitis and other diseases, and even lead to death of patients. IAV belongs to the Orthomyxoviridae family and is an enveloped negative-sense single-stranded ribonucleic acid (RNA) virus. It can be classified into several subtypes based on the antigenic properties of its surface proteins, hemagglutinin (HA) and neuraminidase (NA), including HIN1, H3N2.

IAV is also a zoonotic pathogen with multiple hosts, which can spread among species, and can be reclassified to obtain different virus types, which in turn leads to the emergence of new influenza virus epidemics and widespread epidemics. The Spanish flu outbreak between 1918 and 1919 was the most serious, causing more than 40 million deaths. At present, there are few specific drugs for the treatment of IAV, and drugs with inhibitory effects on IAV have always been the focus of researchers' attention.

The method of using natural chemical components extracted from typical traditional Chinese medicines to inhibit IAV has strong medical value and clinical potential, and conforms to the general research direction of integrated traditional Chinese and western medicine. Compared with the previous medicinal chemistry used to inhibit IAV artificially synthesized small molecules, the natural chemical molecules extracted from plant components can more effectively avoid the possible side effects of chemically synthesized molecules to humans, and have more guarantees in safety and social promotion.

Conessine, a small molecule compound of natural traditional Chinese medicine, is a steroid alkaloid isolated from Holarrhena floribunda (a species within the Holarrhena floribunda genus of the Apocynaceae family), and is one of the important natural active ingredients. Holarrhena floribunda is used in traditional Chinese medicine to primarily treat dysentery and intestinal flatulence, exhibiting antidiarrheal and antipyretic effects. Its functions and indications include clearing heat and relieving cough, detoxifying and killing parasites, astringing the intestines to stop diarrhea, cooling blood to check dysentery, promoting diuresis to alleviate pain. It demonstrates remarkable efficacy in treating conditions such as wind-heat cold, hepatobiliary disorders, gastrointestinal diseases, and diarrhea.

In previous studies, Conessine, a small molecule compound of natural traditional Chinese medicine, is widely used as a highly effective and selective H3 receptor antagonist. At present, it has been found that it mainly has antimalarial activity, anti-dysentery activity, inhibitory activity of efflux pump of Pseudomonas aeruginosa and other antibacterial activities. At the cellular level, Conessine has been found to have the function of regulating autophagy and can inhibit muscle cell death caused by over-activation of the autophagy pathway induced by hydrogen peroxide. Conessine is a mature small molecule drug derived from natural plant components, and has the safe prerequisite to become a good inhibitor of IAV.

However, there are few reports on the antiviral effect of small molecule compound Conessine at present. Therefore, it is of great significance to further develop the antiviral effect of Conessine to solve the global influenza epidemic.

### SUMMARY

An objective of the present disclosure is to provide an application of Conessine in preparing an anti-IAV infection drug. The Conessine is used for treating IAV infection, with good treatment inhibitory effect, and high safety.

In order to achieve the above objective, the present disclosure provides the following technical solutions.

The present disclosure provides an application of Conessine in preparing an anti-IAV infection drug.

Further, the Conessine has a structure as shown in Formula (I):

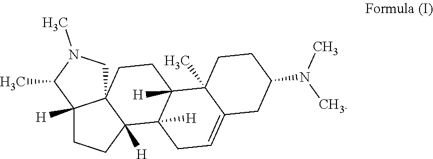

Further, the Conessine can inhibit infection and replication of IAV.

Further, the anti-IAV infection drug can be added with a pharmaceutically acceptable carrier.

Further, a dosage form of the anti-IAV infection drug is one of powder, solution, capsule, granule, tablet, emulsion and suspension.

Compared with the related art, the application of Conessine in preparing an anti-IAV infection drug has the following beneficial effects.
(1) In the present disclosure, the small molecule Conessine is used to inhibit the IAV, and the IC₅₀ value of the inhibitory effect of Conessine is detected. The results show that Conessine has a strong inhibitory effect on the IAV, IC₅₀=2.07 μM, with a low value and a high inhibitory efficiency. The toxicity of the small molecule Conessine to cells is also detected. It is found that its toxicity to cells is relatively low, CC₅₀=34.11 μM, with a high value, and the selection index (SI) is much higher than the safe range, which is more secure for the safety and effectiveness of Conesine as an IAV inhibitor.
(2) In the present disclosure, the natural chemical component-small molecule Conessine extracted from plants is used for inhibiting IAV, which can effectively avoid the side effects of chemically synthesized molecules and has higher safety. Moreover, the small molecule Conessine has a high inhibitory efficiency against IAV. Conessine can be used for treating respiratory infection, pneumonia, encephalitis and other diseases caused by IAV, providing a new effective therapeutic drug for the global influenza epidemic.

### BRIEF DESCRIPTION OF THE DRAWINGS

To explain the technical solutions of examples in the present disclosure or in the related art more clearly, the accompanying drawings required in the description of the examples are introduced briefly below. Obviously, the drawings in the following description are only some examples of the present disclosure, and other drawings can be obtained according to these drawings without creative efforts for those ordinary skilled in the art.

FIG. 1 shows a cytotoxicity curve of Conessine and an inhibition rate curve of IAV in Test Example 1.

### DETAILED DESCRIPTION

Examples of the present disclosure will be described in detail below, which are intended to explain the present disclosure, but not to be construed as limiting the present disclosure. In case the specific techniques or conditions are not specified in the examples, the techniques or conditions described in the literature in this field or the product specification shall be followed. All reagents or instruments used are conventional products that can be obtained through market purchase if the manufacturer is not indicated.

The manufacturer of the small molecule Conesine (alias: Conesine, Wrightine, Diephedrine, Roquessine, Neriine, and Konessin) used in the following examples of the present disclosure is targetmo, and the product number is T20712 CAS546-06-5.

The small molecule Conessine of the present disclosure belongs to steroid alkaloid, has a molecular formula of C₂₄H₄₀N₂, a molecular weight of 356.59, and has a structure as shown in Formula (I):

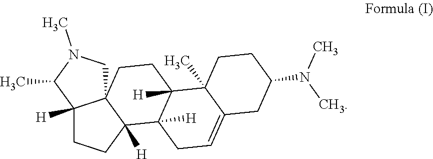

### Example 1

In Example 1 of the present disclosure, screening of drugs for inhibiting IAV was performed, including the following steps.

Preliminary screening was performed on small molecules with IAV inhibition in a small molecule drug library:

The concentration of small molecule drugs in the small molecule library is 10 mM (in ethanol). The screened IAV is linked to the gene expressing Gaussia Luciferase (Gluc) luciferase. The infection and replication of the virus in the host cell can be obtained by detecting the fluorescein value after infecting the cells.

The small molecule Conessine in the small molecule library was diluted 1000 times to 10 μM in Dulbecco's Modified Eagle Medium (DMEM) without serum, and IAV A/PR/8/34 (PR8)-Gluc strain was added to the mixture. The original viral titer was 1.0×10⁵, and the final viral concentration was diluted 100 times. After mixed infection of Madin-Darby canine kidney (MDCK) cells for 24 h, the cells were detected for fluorescein value detection (Gaussia-Lumi™ Gaussian luciferase reporter gene detection kit Biyuntian Bio), and at the same time, the positive control group and the negative control group with only IAV virus infection and virus-free pure cells were added for fluorescein value detection. Finally, it was found that the fluorescein value value of Conessine was close to that of pure cells. Meanwhile, the fluorescein values of most other small molecules were close to those of the control group with IAV infected cells only. It can be seen that the inhibition rate of Conessine on IAV is close to 100%, and Conessine can be used to prepare drugs for the treatment of IAV infection.

### Example 2

In Example 2 of the present disclosure, a tablet anti-IAV infection drug was prepared by Conessine, including the specific preparation steps that:

Conessine was taken, conventional auxiliary materials for preparing tablets were added, and a mixture was mixed evenly for tablet pressing.

### Example 3

In Example 3 of the present disclosure, a granule anti-IAV infection drug was prepared by Conessine, including the specific preparation steps that:

Conessine was taken, conventional auxiliary materials for preparing granules were added, and a mixture was mixed evenly for sub-package.

### Example 4

In Example 4 of the present disclosure, an aerosol anti-IAV infection drug was prepared by Conessine, including the specific preparation steps that:

Conessine was taken, and conventional auxiliary materials for preparing aerosols were added, including propellants and various additives.

### Example 5

In Example 5 of the present disclosure, a capsule anti-IAV infection drug was prepared by Conessine, including the specific preparation steps that:

Conessine was taken, conventional auxiliary materials for preparing capsules were added, and a mixture was mixed evenly, quantitatively packed into capsules with gelatin as the main material and sealed.

### Test Example 1

In Test Example 1 of the present disclosure, the inhibitory effect of Conessine on IAV and cytotoxicity thereof are detected, and the specific methods are as follows.

### (1) Detection of Inhibition Effect

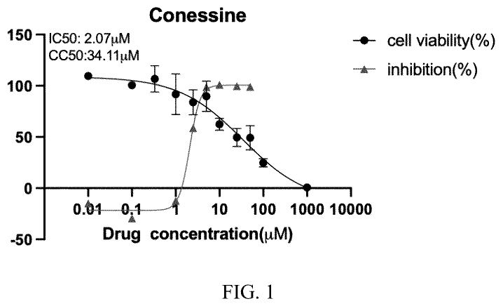

Small molecule Conessine was diluted in DMEM without serum to concentrations of 50 μM, 25 μM, 10 μM, 5 μM, 2.5 μM, 1 μM, 0.1 μM, and 0.01 μM, and different concentrations of Conessine were taken to treat MDCK cells. At the same time, MDCK cells were infected with IAV-Gaussia luciferase (Gluc). After 24 h of infection, luciferase was detected and the value was read, and the curve analysis of the results was performed to finally obtain the small molecule concentration value IC₅₀ with half of the inhibition rate. The results are shown in FIG. 1.

### (2) Cytotoxicity Detection

The small molecule Conessine was diluted in DMEM without serum to concentrations of 1000 μM, 100 μM, 50 μM, 25 μM, 10 μM, 5 μM, 2.5 μM, 1 μM, 0.33 μM, 0.1 μM, and 0.01 μM, the cells were incubated with different concentrations of small molecule Conessine, and five groups of cells under the same conditions were set for average control. The cytotoxicity was measured by CCK8 kit (Enhanced Cell Counting Kit-8 Biyuntian Bio), the cytotoxicity curve of the small molecule was drawn, and the curve analysis of the results was performed to finally obtain the small molecule concentration value CC50 with half of the cytotoxicity. The results are shown in FIG. 1.

It can be seen from FIG. 1 that Conessine has strong inhibitory effect on IAV, IC₅₀=2.07 μM, with a low value. CC₅₀=34.11 μM, with high value, low toxicity to cells, and SI far above the safe range, which ensures the safety and effectiveness of Conessine as the IAV inhibitor.

The above-mentioned examples are only the preferred examples of the present disclosure, it is to be pointed out that for those those ordinary skilled in the art, several improvements and embellishments can be made without departing from the principle of the present disclosure, and these improvements and embellishments are also regarded as the protection scope of the present disclosure.

1. An application of Conessine in preparing an anti-influenza A virus (IAV) subtype H1N1 infection drug;
wherein the Conessine has a structure as shown in Formula (I):

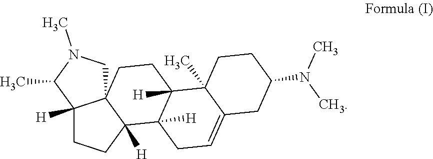

2.

3. The application of Conessine in preparing an anti-IAV subtype H1N1 infection drug according to claim 1, wherein the Conessine is capable of inhibiting an infection and a replication of LAV subtype H1N1.

4. The application of Conessine in preparing an anti-IAV subtype H1N1 infection drug according to claim 3, wherein the anti-IAV subtype H1N1 infection drug comprises a pharmaceutically acceptable carrier.

5. The application of Conessine in preparing an anti-IAV subtype H1N1 infection drug according to claim 4, wherein a dosage form of the anti-IAV subtype H1N1 infection drug is one of powder, solution, capsule, granule, tablet, emulsion or suspension.
